(19) United States
(12) Patent Application Publication
(10) Pub. No.: US 2022/0226040 A1
(43) Pub. Date: Jul. 21, 2022

(54) HIGH VOLTAGE STEERABLE CATHETER

(71) Applicant: Oscor Inc., Palm Harbor, FL (US)

(72) Inventors: Thomas P. Osypka, Palm Harbor, FL (US); Timothy Searfoss, New Port Richey, FL (US)

(73) Assignee: Oscor Inc., Palm Harbor, FL (US)

(21) Appl. No.: 17/537,863

(22) Filed: Nov. 30, 2021

Related U.S. Application Data
(60) Provisional application No. 63/137,855, filed on Jan. 15, 2021.

(51) Int. Cl. A61B 18/14; A61M 25/01

(52) U.S. Cl. CPC A61B 18/1492; A61B 2018/00083; A61M 25/0136; A61M 25/0147

(57) ABSTRACT

A steerable high voltage catheter is disclosed that includes a handle assembly, a shaft extending distally from the handle assembly, an electrode assembly operatively associated with a distal end portion of the shaft for delivering high voltage energy to cardiac tissue, a plurality of conductive wires extending from the handle assembly, through the shaft to the electrode assembly to carry high voltage energy thereto, and a rotatable steering mechanism within the handle assembly that is adapted and configured to deflect the distal end portion of the tubular shaft.

### CROSS-REFERENCE TO RELATED APPLICATION

The subject application claims the benefit of priority to U.S. Provisional Patent Application Ser. No. 63/137,855 filed Jan. 15, 2021, the disclosure of which is incorporated herein by reference in its entirety.

### BACKGROUND OF THE INVENTION

### 1. Field of the Invention

The subject invention is directed to a catheter for cardiac procedures, and more particularly, to a high voltage wiring layout for a steerable catheter used in cardiac ablation or cardioversion procedures.

### 2. Description of Related Art

Cardiac arrhythmia or an abnormal heart rhythm can cause problems such as fainting, stroke, heart attack, and even sudden cardiac death. Treatments for the condition include electrical cardioversion and cardiac ablation. In a cardioversion procedure, a high-energy shock is sent to the heart to reset a normal rhythm. In a cardiac ablation procedure, an electrophysiology (EP) catheter is deployed intravenously to map out and ablate the tissue in the heart that is allowing the incorrect electrical signals to cause an abnormal heart rhythm.

The catheter devices utilized in these procedures have conductors that y high voltage energy connected electrodes that deliver high energy shocks to heart tissue for either cardioversion or pulsed ablation therapy. It is necessary to insulate these high voltage wires to prevent shorts from occurring during a procedure. In the past, manufacturers have placed polyimide tubing over each wire in the catheter shaft to prevent arcing. However, wiring layouts are often complex and polyimide tubing cannot effectively cover all of the wires inside the catheter, making them susceptible to shorts. Therefore, there is a need in the art for a solution to this problem, so as to provide a safer product.

### SUMMARY OF THE DISCLOSURE

The subject invention is directed to a new and useful high voltage catheter for use in cardiac ablation or cardioversion procedures, which includes a handle assembly, an elongated tubular shaft extending distally from the handle assembly and an electrode assembly operatively associated with a distal end portion of the tubular shaft for delivering high voltage energy to cardiac tissue. A plurality of conductive wires extend from the handle assembly through the tubular shaft to the electrode assembly to carry high voltage energy thereto.

Preferably, each conductive wire has a wire gauge of AWG 40, is formed from a nickel based alloy and is coated with insulation having a thickness that is greater than a conventional wire insulation thickness to provide enhanced dielectric performance. More particularly, each conductive wire is coated with insulation having a thickness that is at least 2 to 3 times greater than a conventional wire insulation thickness. Consequently, the insulation on each conductive wire is rated for dielectric performance to 10 kV.

The electrode assembly includes a plurality of longitudinally spaced apart electrode rings formed from a platinum iridium material for electrophysiological mapping, electrical cardioversion and/or pulsed ablation. A respective conductive wire from the plurality of conductive wires is laser welded to an inner diameter of each electrode ring. The handle assembly is operatively associated with a set of high voltage connectors that are rated to 7 kV, and conductive wires extend between the handle assembly and the high voltage connectors.

The handle assembly includes a rotatable bi-directional steering mechanism that is adapted and configured to deflect the distal end portion of the tubular shaft. A pair of non-conductive steering cables extend from the rotatable bi-directional steering mechanism in the handle assembly to the distal end portion of the shaft to facilitate deflection by pulling in either direction. Preferably, the non-conductive steering cables are formed from Kevlar® thread. The rotatable bi-directional steering mechanism has a circular body, and the steering cables are anchored to the circular body on diametrically opposed bobbins by respective set screws.

The subject invention is also directed to a steerable catheter, which includes a handle assembly, an elongated tubular shaft extending distally from the handle assembly, and a rotatable bi-directional steering mechanism in the handle assembly that is adapted and configured to deflect the distal end portion of the tubular shaft, wherein a pair of non-conductive steering cables extend from the steering mechanism to the distal end portion of the shaft.

The subject invention is also directed to a steerable high voltage catheter, which includes a handle assembly, a tubular shaft extending distally from the handle assembly, an electrode assembly associated with a distal end portion of the tubular shaft for delivering high voltage energy to cardiac tissue, wherein the electrode assembly includes a plurality of longitudinally spaced apart electrode rings formed from a platinum iridium material.

The steerable high voltage catheter further includes a plurality of conductive wires extending from the handle assembly through the tubular shaft to the electrode assembly to carry high voltage energy thereto, wherein each conductive wire has a wire gauge of AWG 40, is formed from a nickel based alloy is coated with insulation having a thickness that is at least 2 to 3 times greater than greater than a conventional wire insulation thickness.

The steerable high voltage catheter also includes a rotatable bi-directional steering mechanism within the handle assembly that is adapted and configured to deflect the distal end portion of the tubular shaft, wherein a pair of non-conductive steering cables extend from the steering mechanism to the distal end portion of the shaft, and wherein each steering cables is formed from Kevlar® thread.

These and other features of the steerable high voltage catheter of the subject invention will become more readily apparent to those having ordinary skill in the art to which the subject invention appertains from the detailed description of the preferred embodiments taken in conjunction with the following brief description of the drawings.

### BRIEF DESCRIPTION OF THE DRAWINGS

So that those skilled in the art will readily understand how to make and use the steerable high voltage catheter of the subject invention without undue experimentation, preferred embodiments thereof will be described in detail herein below with reference to the figures wherein:

FIG. 1 is a perspective view of the steerable high voltage catheter of the subject invention, with the steering mechanism in the handle assembly and the electrode assembly at the distal end portion of the shaft removed for ease of illustration;

FIG. 2 is a perspective view of the electrode assembly at the distal end portion of the shaft;

FIG. 3 is partial perspective view of the electrode assembly shown in FIG. 2, wherein the material of the shaft is removed for ease of illustration;

FIG. 4 is an enlarged perspective view of a portion of an insulated conductive wire; electrical conductor;

FIG. 5 is an enlarged perspective view of the handle assembly shown in FIG. 1, illustrating the plurality of conductive wires leading into the shaft; and

FIG. 6 is partial top plan view of the handle assembly illustrating to the rotatable steering mechanism, with the steering cables anchored thereto.

### DETAILED DESCRIPTION OF THE PREFERRED EMBODIMENTS

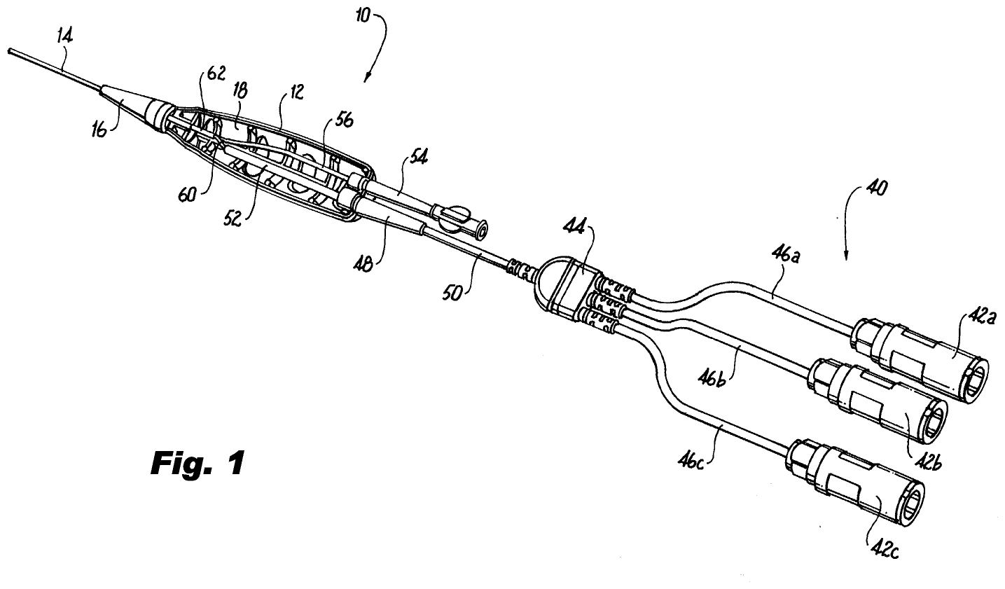

Referring now to the drawings wherein like reference numerals identify similar structural features of the subject invention, there is illustrated in FIG. 1 a steerable high voltage catheter for use in cardiac ablation or cardioversion procedures, which is designated generally by reference numeral 10.

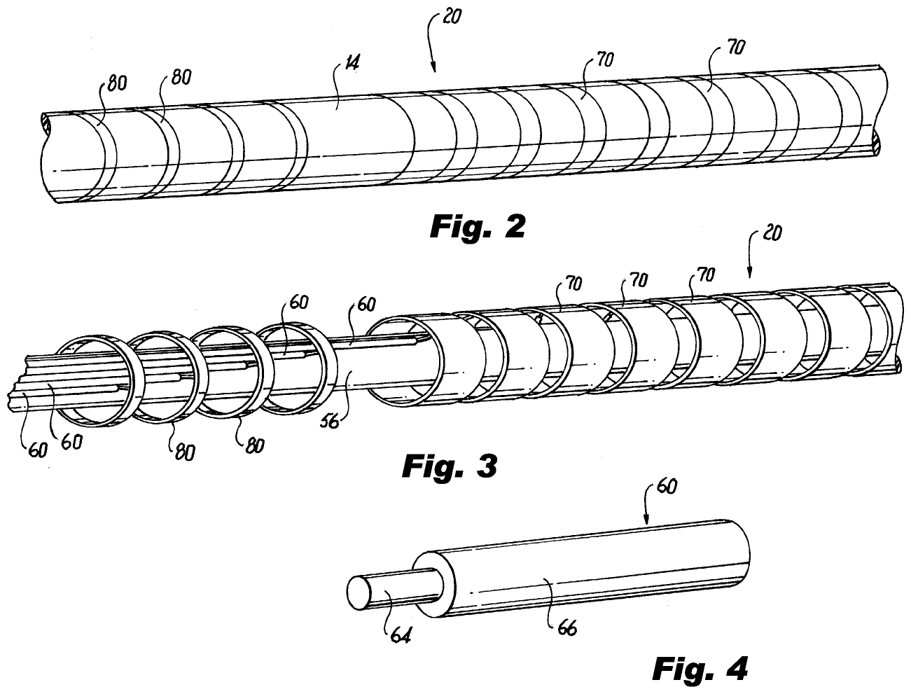

The catheter 10 includes a proximal handle assembly 12 and an elongated tubular shaft 14 extending distally from a nose piece 16 associated with a distal end portion of the handle assembly 12. As best seen in FIG. 2, an electrode assembly 20 is operatively associated with a distal end portion of the tubular shaft 14 for delivering high voltage energy to cardiac tissue. The electrode assembly 20, which will be described in greater detail below with respect to FIG. 3, includes a plurality of electrode rings 70, 80 for electrophysiological mapping, electrical cardioversion and/or pulsed ablation.

With continuing reference to FIG. 1, a high voltage connector assembly 40 is operatively associated with a proximal end portion of the handle assembly 12 for connection with power supply cables (not shown). The connector assembly 40 includes three (3) separate connectors 42a-42c that are each rated to 7 kV. The high voltage connectors 42a-42c extend to a junction coupling 44 by way of respective flexible wire conduits 46a-46c. The junction coupling 44 is connected to a fitting 48 that extends from the proximal end of the housing assembly 12 by way of a flexible conduit 50.

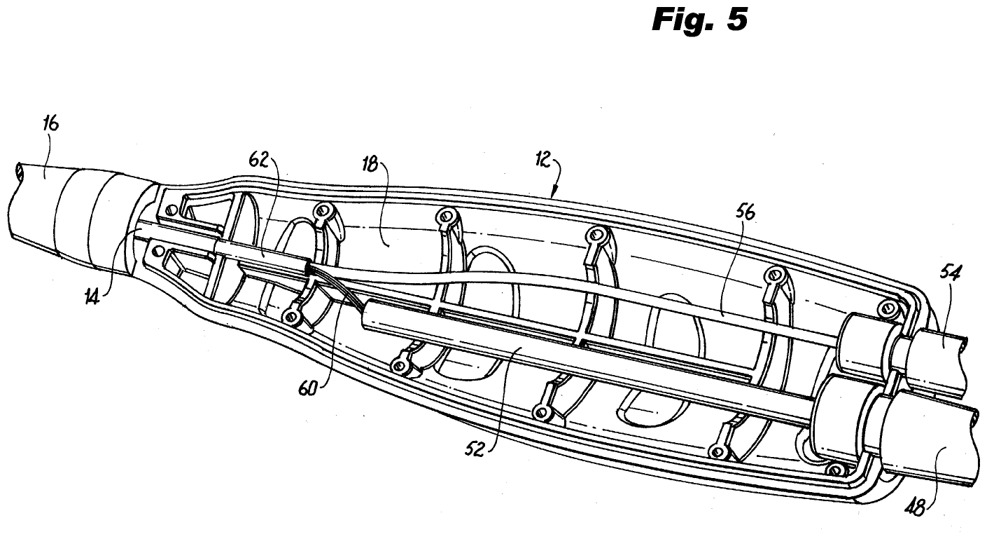

The fitting 48 leads to a tubular conduit 52 located within the interior cavity 18 of handle assembly 12 for accommodating the plurality of high voltage insulated conductive wires 60 that emanate from the connector assembly 40, as best seen in FIG. 5. The insulated conductive wires 60 extend into an elongated guide tube 62 that leads from the interior cavity 18 of handle assembly 12, through the elongated tubular shaft 14 of catheter 10.

A luer fitting 54 also extends from the proximal end portion of the handle assembly 12 for connecting the handle assembly 12 to a source of suction or irrigation, for example. An irrigation/suction conduit 56 extends from the fitting 54, through the interior cavity 18 of handle assembly 12 to the elongated guide tube 62. The conduit 56 preferably communicates with one or more ports (not shown) associated with a distal end portion of the elongated shaft 14 of catheter 10.

Referring now to FIG. 3, the plurality of insulated conductive wires 60 extend from the handle assembly 12 through the tubular shaft 14 to the electrode assembly 20, in order to carry high voltage energy thereto. Preferably, the insulated conductive wires 60 are Nickel based wires, such as, for example, Nichrome wires comprised primarily of nickel and chromium, which exhibit low resistivity characteristics. These conductive wires 60 are preferably 40 gauge wire (AWG 40), although other wire gauges could be employed. Also, in accordance with the subject invention, each insulated conductive wire 60 is coated with insulation having a thickness or build that is greater than a conventional wire insulation thickness to provide enhanced dielectric performance. More particularly, with reference to FIG. 4, each insulated conductive wire 60 includes a conductor 64 coated with insulation 66 having a thickness or build that is at least 2 to 3 times greater than a conventional wire insulation thickness. Consequently, the insulation 64 of each conductive wire 60 is rated for dielectric performance to 10 kV.

Those skilled in the art will appreciate that insulation thickness, or build, is the measurement of coating that has been added to the circumference of a wire. It can be determined by taking the total diameter of the conductive wire and the insulation together, and then subtracting the diameter of just the wire from the total diameter. For 40 gauge wire (AWG 40), which has a nominal diameter of 0.00314 inches, the insulation build typically ranges from 0.0002 to 0.0006 inches, where larger insulation builds are used to make the wire stronger or to offer more protection. By way of comparison, in accordance with the subject invention, the conductive wires 60 have an insulation thickness of about approximately 0.0015 inches, providing enhanced dielectric performance rated to 10 kV.

Referring back to FIG. 3, the electrode assembly 20 includes a plurality of longitudinally spaced apart proximal electrode rings 70 formed from a platinum iridium material and a plurality of smaller distal electrode rings 80. These electrodes are adapted and configured for electrophysiological mapping, electrical cardioversion and/or pulsed ablation to facilitate the treatment of cardiac arrhythmia conditions. A respective conductive wire 60 is laser welded to an inner diameter of each electrode ring 70, 80. An adhesive may be used additionally. Because these insulated conductive wires have such a substantial insulation build, there is no need to include a secondary covering or wrap over the wires, as is typical in prior art devices.

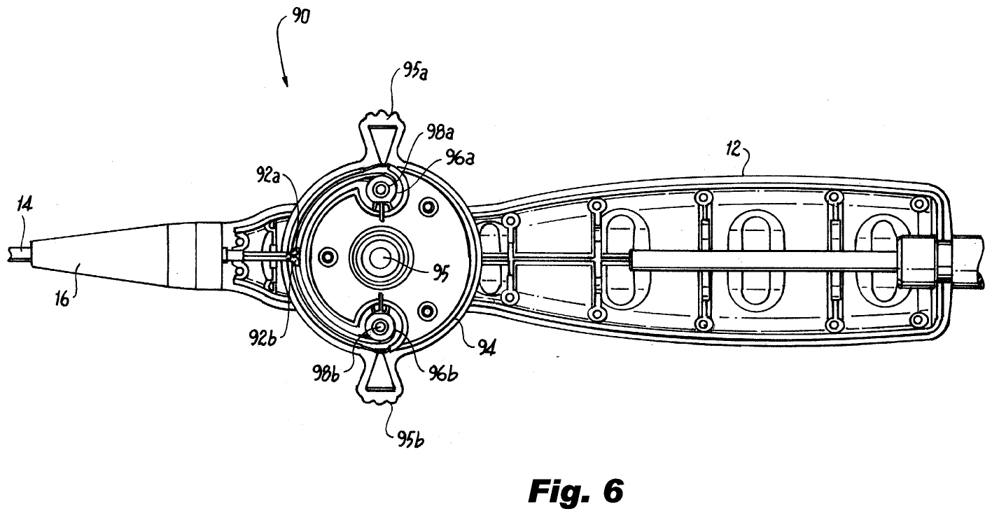

Referring now to FIG. 6, the handle assembly 12 includes a rotatable bi-directional steering mechanism 90 that is adapted and configured to deflect or otherwise steer the distal end portion of the tubular shaft 14 for improved intravascular placement. A pair of non-conductive steering cables 92a and 92b extend from the rotatable bi-directional steering mechanism 90 in the handle assembly 12 to the distal end portion of the shaft 14 to facilitate deflection of the distal end portion of the shaft by pulling in either direction.

Preferably, the non-conductive steering cables 92a and 92b are formed from Kevlar® thread, which is a heat resistant para-aramid synthetic fiber with molecular structure of many inter-chain bonds that provide strength. Because the steering cables 92a and 92b are formed from Kevlar thread, as opposed to a metal wire, there is no risk that they might puncture through the catheter wall if they break loose from their anchor points in the distal end portion of the shaft 14.

The rotatable bi-directional steering mechanism 90 has a circular body 94 supported on a central pivot 95, and the steering cables 92a and 92b are anchored to the circular body 94 on diametrically opposed bobbins 96a and 96b by respective set screws 98a and 98b. The circular body 94 includes diametrically opposed steering flanges 95a and 95b for manually rotating the steering mechanism 90.

While the subject disclosure has been shown and described with reference to preferred embodiments, those skilled in the art will readily appreciate that changes and/or modifications may be made thereto without departing from the scope of the subject disclosure.

What is claimed is:

1. A high voltage catheter, comprising
a) a handle assembly;
b) an elongated tubular shaft extending distally from the handle assembly;
c) an electrode assembly operatively associated with a distal end portion of the tubular shaft for delivering high voltage energy to cardiac tissue; and
d) a plurality of conductive wires extending from the handle assembly, through the tubular shaft to the electrode assembly to carry high voltage energy thereto, wherein each conductive wire is coated with insulation having a thickness that is greater than a conventional wire insulation thickness to provide enhanced dielectric performance.

2. The high voltage catheter recited in claim 1, wherein each conductive wire has a wire gauge of AWG 40, is formed from a nickel based alloy and is coated with insulation having a thickness that is at least 2 to 3 times greater than a conventional wire insulation thickness.

3. The high voltage catheter recited in claim 1, wherein the insulation on each conductive wire has a thickness of about 0.0015 inches and is rated for dielectric performance to 10 kV.

4. The high voltage catheter recited in claim 1, wherein the electrode assembly includes a plurality of longitudinally spaced apart electrode rings formed from a platinum iridium material, and wherein a respective conductive wire from the plurality of conductive wires is laser welded to an inner diameter of each electrode ring.

5. The high voltage catheter recited in claim 1, wherein the handle assembly is operatively associated with a set of high voltage connectors rated to 7 kV, and wherein the conductive wires extend between the handle assembly and the high voltage connectors.

6. The high voltage catheter recited in claim 1, wherein the handle assembly includes a rotatable bi-directional steering mechanism that is adapted and configured to deflect the distal end portion of the tubular shaft.

7. The high voltage catheter recited in claim 6, wherein a pair of non-conductive steering cables extend from the rotatable bi-directional steering mechanism in the handle assembly to the distal end portion of the shaft.

8. The high voltage catheter recited in claim 7, wherein the non-conductive steering cables are formed from Kevlar® thread.

9. The high voltage catheter recited in claim 6, wherein the rotatable bi-directional steering mechanism has a circular body, and the steering cables are anchored to the circular body on diametrically opposed bobbins by respective set screws.

10. A steerable catheter, comprising
a) a handle assembly;
b) an elongated tubular shaft extending distally from the handle assembly; and
c) a rotatable bi-directional steering mechanism in the handle assembly that is adapted and configured to deflect the distal end portion of the tubular shaft, wherein a pair of non-conductive steering cables extend from the steering mechanism to the distal end portion of the shaft.

11. The steerable catheter recited in claim 10, wherein the non-conductive steering cables are formed from Kevlar® thread.

12. The steerable catheter recited in claim 10, wherein the rotatable bi-directional steering mechanism has a circular body, and the steering cables are anchored to the circular body on diametrically opposed bobbins by respective set screws.

13. The steerable catheter recited in claim 10, wherein an electrode assembly is operatively associated with the distal end portion of the tubular shaft for delivering high voltage energy to cardiac tissue.

14. The steerable catheter recited in claim 13, wherein a plurality of conductive wires extending from the handle assembly, through the tubular shaft to the electrode assembly to carry high voltage energy thereto, wherein each conductive wire is coated with insulation having a thickness that is greater than a conventional wire insulation thickness.

15. The high voltage catheter recited in claim 14, wherein each conductive wire has a wire gauge of AWG 40, is formed from a nickel based alloy and is coated with insulation having a thickness that is at least 2 to 3 times greater than a conventional wire insulation thickness.

16. The steerable catheter recited in claim 14, wherein the insulation on each conductive wire has a thickness of about 0.0015 inches is rated for dielectric performance to 10 kV.

17. The steerable catheter recited in claim 10, wherein the electrode assembly includes a plurality of longitudinally spaced apart electrode rings formed from a platinum iridium material, and wherein a respective conductive wire from the plurality of conductive wires is laser welded to an inner diameter of each electrode ring.

18. The steerable catheter recited in claim 10, wherein the handle assembly is operatively associated with a set of high voltage connectors rated to 7 kV, and wherein the conductive wires extend between the handle assembly and the high voltage connectors.

19. A steerable high voltage catheter, comprising
a) a handle assembly;
b) an elongated tubular shaft extending distally from the handle assembly;
c) an electrode assembly operatively associated with a distal end portion of the tubular shaft for delivering high voltage energy to cardiac tissue, wherein the electrode assembly includes a plurality of longitudinally spaced apart electrode rings formed from a platinum iridium material;
d) a plurality of conductive wires extending from the handle assembly through the tubular shaft to the electrode assembly to carry high voltage energy thereto, wherein each conductive wire has a wire gauge of AWG 40, is formed from a nickel based alloy and is coated with insulation having a thickness that is about 0.0015 inches; and
e) a rotatable bi-directional steering mechanism within the handle assembly that is adapted and configured to deflect the distal end portion of the tubular shaft, wherein a pair of non-conductive steering cables extend from the steering mechanism to the distal end portion of the shaft, an wherein each steering cable is formed from Kevlar® thread.

20. The steerable high voltage steerable catheter recited in claim 19, wherein a conductive wire from the plurality of conductive wires is laser welded to an inner diameter of each electrode ring.
